(19) United States
(12) Patent Application Publication
(10) Pub. No.: US 2024/0301177 A1
(43) Pub. Date: Sep. 12, 2024

(54) IMPROVED METHOD FOR THE PREPARATION OF MICROCRYSTALLINE CELLULOSE

(71) Applicant: NUTRITION & BIOSCIENCES USA 1, LLC, Rochester, NY (US)

(72) Inventors: Rene Kelling, Bomlitz (DE); Olena Vozniuk, Bomlitz (DE); Stefan Junge, Bomlitz (DE); Michael Schreck, Bomlitz (DE); Jing G. Guo, Rochester, NY (US); Matthias R. Sprehe, Bomlitz (DE)

(21) Appl. No.: 18/261,296

(22) PCT Filed: Jan. 12, 2022
(86) PCT No.: PCT/EP2022/050567

§ 371 Date: Jul. 13, 2023

Related U.S. Application Data
(60) Provisional application No. 63/136,709, filed on Jan. 13, 2021.

(51) Int. Cl. C08L 1/04; A61K 47/38; C08B 15/02

(52) U.S. Cl. CPC C08L 1/04; A61K 47/38; C08B 15/02

(57) ABSTRACT

A mill dried microcrystalline cellulose (MCC) is obtained in a process comprising the steps of a) providing MCC having a moisture content of from 20 to 75 percent, based on the total weight of the moist MCC; and b) mill-drying the moist MCC in a single device capable of milling and drying in combination. The process can provide mill-dried microcrystalline cellulose having similar particle size distribution (LEFI, DIFI and EQPC) and moisture content as the corresponding spray-dried microcrystalline cellulose. The process can provide mill-dried microcrystalline cellulose having a higher tapped/untapped bulk density and/or a lower Carr index, while keeping a similar moisture content, as the corresponding spray-dried microcrystalline cellulose.

### FIELD

The present invention relates to a new process for producing dried microcrystalline cellulose (MCC), to MCC producible by the new process and to MCC having new properties.

### INTRODUCTION

Microcrystalline cellulose (MCC) is a purified product which is produced by converting fibrous cellulose into a highly crystalline cellulose by selective hydrolytic degradation of amorphous regions of the fibrous cellulose. The sources for the preparation of MCC can be cellulose pulp from fibrous plants materials such as wood or other cellulosic materials such as cotton from linters, stalks, rags or fabric waste. MCC products are used as binders and disintegrants in pharmaceutical tablets and as suspending liquids in pharmaceutical formulations. MCC is widely used as a binder, gelling agent, thickener, texturizer, stabilizer, emulsifier and as fat replacement in food and beverage applications. Moreover, MCC products find use as for example binders or bulking agents in personal care applications, such as cosmetics and dentifrices, or as a binder, bulking agent, disintegrant or processing aid in cosmetics and dentifrices, in industrial applications such as in paint, in household products such as detergents or bleach tablets, and in agricultural formulations.

The classical method for MCC production, which is still the most common manufacturing method, is acid hydrolysis of purified cellulose as for example disclosed in U.S. Pat. No. 2,978,446.

Other processes for the preparation of microcrystalline cellulose have been developed such as steam explosion (U.S. Pat. No. 5,769,934), reactive extrusion (U.S. Pat. No. 6,228,213), high temperature reaction in a reactor pressurized with oxygen and/or carbon dioxide gas (U.S. Pat. No. 5,543,511).

Following the acidic hydrolysis, microcrystalline cellulose is separated from the reaction mixture to provide a wetcake.

The separated microcrystalline cellulose wetcake is subsequently dried to powdered grades of MCC. A wide range of commercial microcrystalline cellulose products are available, for example under the brand name of Avicel®.

A final step in manufacturing of MCC is drying of the MCC wet-cake. In commercial manufacturing the drying-step is commonly performed by spray-drying. The desired commercial grades of MCC are obtained by varying and controlling the spray drying conditions in order to manipulate the degree of agglomeration (particle size distribution) and moisture content of the MCC product. [G. Thorens, Int. J. Pharm. (2015), 490, 47-54; G. Thorens, Int. J. Pharm. (2014), 473, 64-72)]. However, spray-drying can only be performed on slurries having a high water content and therefore the MCC wet-cake must be diluted with water prior to spray-drying.

An aqueous slurry of MCC for spray drying typically comprises ca. 10%-20% (w/w) MCC and 80%-90% (w/w) water. Spray drying of MCC is therefore associated with very high water and energy expenses due to the large amount of water to be added and subsequently to be evaporated. Furthermore, spray-drying equipment is very costly.

M. Tomar et al., Int. J. Pharm. Sci. Res. (2018), Vol. 9(4), 1545-1554, disclose several alternative methods for drying MCC wet cake that has been produced by acid hydrolysis of dissolving grade wood pulp and neutralization by washing with water and ammonia. For spray drying water is added to the MCC wet cake to make a suspension which is then dried with the help of a spray dryer. For spin flash drying the MCC wet cake is first broken down into small pieces with a mixer, followed by drying of the material with a spin flash dryer, i.e. the break down of wet cake and the subsequent drying of wet cake takes place in two separate unit operations and in separate process devices. For bulk flash drying the MCC wet cake is also first crushed down into small pieces with help of a mixer, followed by drying with a bulk drier. As generally known by the skilled artisans, in spin flash dryers and bulk flash dryers material is dried but not comminuted. Generally, there is a common understanding among commercial MCC producers that spray-drying of MCC inherently leads to technically superior qualities of MCC, such as products of desired morphology, viscosity and performance.

Thus, there is a need for a cost-efficient method for drying aqueous microcrystalline cellulose wet-cake. Particularly there is a need for cost-efficient methods for drying aqueous microcrystalline cellulose wet-cake in an industrial scale while retaining favourable properties of the dried microcrystalline cellulose product, such as favourable particle size, particle morphology, bulk density, flowability and moisture content.

### SUMMARY

In one aspect the present invention relates to a process for producing mill dried microcrystalline cellulose (MCC) comprising the steps of a) providing MCC having a moisture content of from 20 to 75 percent, based on the total weight of the moist MCC, and b) mill-drying the moist MCC in a single device capable of milling and drying in combination.

In another aspect the present invention relates to the microcrystalline cellulose producible by the process described above.

In yet another aspect the present invention relates to microcrystalline cellulose having a moisture content of less than 20% by weight, based on the total weight of the microcrystalline cellulose including moisture, and having an untapped bulk density of at least 350 g/L or a Carr Index of up to 28 or both.

In a further aspect the present invention relates to the use of a microcrystalline cellulose according to the embodiments above in food or beverage applications, in pharmaceutical applications or in personal care applications.

The present inventors have found that microcrystalline cellulose having a moisture content of 20-75%, based on the total weight of the moist MCC, can be subjected to drying and milling in combination in a single mill-drying device to provide mill dried MCC. According to the process of the present invention mill dried MCC can be produced that has particle sizes (LEFI, DIFI and/or EQPC) that are taylor-made according to the needs of the particular end-uses. The process of the present invention enables control of the morphology of the mill dried MCC and the production of mill dried MCC having a wide variety of particle sizes.

In one aspect of the invention, mill dried MCC can be produced that exhibits comparable particle sizes (LEFI, DIFI and/or EQPC) as microcrystalline cellulose prepared by traditional spray-drying of microcrystalline cellulose slurry having a water content of ca. 80-90%. Particularly, mill-drying of microcrystalline cellulose according to the process of the present invention can provide mill-dried microcrystalline cellulose having similar particle sizes (LEFI, DIFI and/or EQPC) and moisture content as the corresponding spray-dried microcrystalline cellulose prepared by traditional spray-drying of microcrystalline cellulose slurry having a water content of ca. 80-90%. In another aspect of the invention, mill-dried MCC can be produced having substantially larger or substantially smaller particle sizes than microcrystalline cellulose prepared by traditional spray-drying of microcrystalline cellulose slurry having a water content of ca. 80-90%.

Moreover, mill-drying of microcrystalline cellulose according to the process of the present invention is suitable to provide mill-dried microcrystalline cellulose having a higher tapped/untapped bulk density and/or a lower Carr index, while keeping a similar moisture content as the corresponding spray-dried microcrystalline cellulose. This is advantageous because lower Carr index results in improved powder flow and higher bulk density results in improved packaging, storage and transportation of the dried MCC, such as reduced packaging and transportation costs. Surprisingly, the process of the present invention enables the production of mill dried MCC that exhibits comparable particle sizes (LEFI, DIFI and/or EQPC) as well as improved bulk density and/or lower Carr index compared to microcrystalline cellulose prepared by spray-drying.

The present invention allows for drying of a non-diluted wet-cake of MCC as opposed to traditional spray-drying of MCC which requires dilution of the MCC wet-cake to a moisture content of ca. 80-90%. The reduced water content of the moist MCC leads to reduction in energy-consumption for drying, i.e., the present invention leads to a substantially reduced consumption of water and substantial energy savings for the drying of MCC. In addition to reducing water expenses and energy expenses for drying the MCC, the mill-drying equipment required for performing the present invention is generally cheaper and less space-consuming than spray-dying equipment for drying MCC, leading to reduced capital investment for performing the present invention, as compared to the traditional spray-drying method.

### DETAILED DESCRIPTION OF THE INVENTION

Microcrystalline cellulose (MCC) is a white, odorless, tasteless, relatively free flowing crystalline powder. It is a purified, partially depolymerized cellulose obtained by subjecting alpha cellulose obtained as pulp from fibrous plant material to hydrolytic degradation, typically with mineral acids. Suitable plant material includes, for example, wood pulp such as bleached sulfite and sulfate pulps, corn husks, bagasse, straw, cotton, cotton linters, flax, kemp, ramie, fermented cellulose, etc. During acidic hydrolysis the amorphous regions (or paracrystalline regions) of the cellulosic fibril are selectively hydrolysed while the crystalline regions remain intact, whereby highly crystalline particulate cellulose consisting mainly of crystalline aggregates (MCC) are obtained. The degree of polymerization (DP, the number of anhydroglucose units in the cellulose chain) decreases during the acid hydrolysis and the rate of hydrolysis slows to a certain level-off degree of polymerization (LOPD), typically 200-300. The MCC is separated from the reaction mixture and washed to remove degraded by-products.

After the washing step the MCC wet cake generally has a moisture content of from 35 to 70 percent, typically from 45 to 60 percent, based on the total weight of the moist MCC. Preferred washing liquors generally are water, brine, or organic solvents in admixture with water, such as aqueous mixtures of isopropanol, ethanol or methanol. More preferred washing liquors generally are water or brine. Preferably MCC obtained directly after hydrolysis, washing and optionally cooling is used as a starting material for the present invention. MCC is generally washed at a temperature of from 10 to 80° C., preferably from 15 to 50° C. A solvent-moist, preferably a water-moist mass is obtained after washing and separating the MCC from the washing liquor. Separating MCC from a suspension can be carried out in a known way, such as centrifugation. The resulting wet mass is referred to in the art by several names, including hydrolyzed cellulose, hydrolyzed cellulose wetcake, level-off DP cellulose, microcrystalline cellulose wetcake or simply wetcake.

In the process of the present invention the mill-drying of MCC is conducted in a single device that is capable of milling and drying in combination. Such a device is herein designated as “mill-drying device”. In mill-drying devices milling and drying is done in combination, preferably at least partially simultaneously. Mill-drying devices are clearly distinct in function and design from devices that only serve for drying of material. E.g., the energy input into the drying devices essentially consists of thermal energy. However, mechanical energy and thermal energy are both put into mill-drying devices to a significant degree. The term “mechanical energy” as used herein means the energy, typically the electrical energy, that is required to put and keep the mill-drying device in operation, e.g., in rotational motion. The term “thermal energy” is the energy provided by the pre-heated drying gas that is fed into mill-drying device. In the process of the present invention, the mill-drying device is typically operated at an input of mechanical energy of from 2 to 100 percent, preferably from 5 to 50 percent, more preferably from 7 to 31 percent, based on the total of mechanical and thermal energy input.

A mill-drying device useful in the process of the present invention typically comprises a mill-drying chamber which is equipped with one or more inlets for the moist MCC and gas and with one or more grinding inserts, such as grinding pins, rods, bars, plates or disks. The grinding inserts are generally in movement, preferably in rotational movement, when the mill-drying chamber is in operation and accomplish milling of the MCC by impact and/or shearing. Drying is typically accomplished with a combination of hot gas and mechanical energy. Hot air or hot nitrogen gas can be used. The hot gas and the moist MCC can be fed via separate inlets into the mill-drying chamber, typically hot gas from the bottom and wet product at a side entrance via a feed screw system connected to the mill-drying chamber. Alternatively, the moist MCC can be fed into the gas stream and subsequently via the gas stream into the mill-drying chamber. Depending on the position of the inserts in the mill-drying chamber, the moist MCC can first be partially dryed before it is milled, or the moist MCC can first be partially milled before it is dried, or milling and drying can be conducted simultaneous. However, it is essential that milling and drying is conducted in a single device wherein milling and drying is done in combination.

Mill-drying of the moist MCC can be conducted in a known mill-drying device, for example in an impact mill, preferably a gas-swept impact mill, more preferably an air-swept impact mill, wherein MCC is subjected to an impacting and/or shearing stress as well as to drying.

Particle size, particle morphology, bulk density and flowability of the mill dried MCC can be controlled and/or adjusted by the design and/or operation of the mill-drying device, such as the type and number of grinding inserts like grinding pins, rods, bars, plates or disks or the circumferential speed of the mill-drying chamber. The larger the number of grinding inserts is in a given mill-drying chamber and/or the higher the circumferential speed of a given mill-drying chamber is, the smaller are generally the median particle sizes of the mill dried MCC, such as the median LEFI, DIFI and EQPC described further below. Preferred designs and operations of the mill-drying device are described in more detail below and in the examples.

Preferred air-swept impact mills are Ultra Rotor mills (Altenburger Maschinen Jaeckering, Germany), Contra-Selector PPS (PALLMANN Maschinenfabrik GmbH & Co. KG, Germany), or Turbofiner PLM mills (PALLMANN Maschinenfabrik GmbH & Co. KG, Germany). Gas classifier mills are also useful air-swept (gas-swept) impact mills, for example, the Hosokawa Alpine Air Classifier mill—ZPS Circoplex Hosokawa Micron Ltd., Cheshire, England. Other preferred mill-drying devices are flash mill dryers; they are commercially available, for example from Hosokawa under the trademark Drymeister (DMR). Other suitable mills and mill-type dryers are, for example hammer mills, screen-type mills, pin mills, or centrifugal impact mills, disk mills, or preferably classifier mills.

Air or nitrogen gas can be used for drying. In the process of the present invention the gas fed into the mill-drying device, more specifically the mill-drying chamber of the mill-drying device, typically has a temperature of 180° C. or less, preferably 170° C. or less, and in some embodiments of the invention of 150° C. or less, such as 130° C. or less, or even 110° C. or less. Typically, the gas fed into the mill-drying device has a temperature of 50° C. or more, preferably of 60° C. or more, such as 65° C. or more. A gas stream having the above-mentioned temperature can be created in various ways. In one embodiment of the invention a fresh gas stream having the desired temperature can be fed into the mill-drying device. In another embodiment of the invention a recycled gas stream having the desired temperature is fed into the mill-drying device. For example, a gas stream can be separated from the ground and dried MCC, and the resulting solid-free gas stream, or a portion thereof, can be cooled in a cooling system, e.g., using water as coolant. This resulting cooled gas stream can be fed into the mill-drying device. Alternatively, the entire amount of cooled gas can be re-heated, e.g. in a natural gas burner. To bring the re-heated gas to the desired temperature for feeding into the mill-drying device, a separate stream of cold gas can be combined with the hot gas stream before feeding the gas stream into the mill-drying device.

The gas and the moist MCC stream are generally fed via separate inlets into the mill-drying chamber, typically gas from the bottom and moist MCC at a side entrance via a feed screw system connected to the mill-drying chamber resulting in an upward flow of MCC and gas, while MCC is being contacted with one or more grinding inserts, such as grinding pins, rods, bars, plates or disks inside the Imill-drying chamber. Alternatively, the moist MCC can be fed into the gas stream and subsequently via the gas stream into the mill-drying chamber.

In a further embodiment, superheated vapor of a solvent, such as superheated steam, or a steam/inert gas mixture or a steam/air mixture can be used as heat-transfer gas and transport gas, as described in more detail in European Patent Applications EP 0 954 536 A1 and EP 1 127 910 A1.

The microcrystalline cellulose which is provided for mill drying in a mill-drying device has a moisture content of from 20 to 75 percent, preferably from 30 to 75%, such as from 40 to 75%, such as from 45 to 75%, more preferably from 50 to 70%, such as from 55 to 70%, such as from 60 to 70%, such as from 62 to 68%, based on the total weight of the moist MCC. The moisture content is measured as the loss on drying. The loss on drying is determined according to USP (United States Pharmacopeia) 35<731>‘Loss On Drying’.

In one embodiment of the invention, microcrystalline cellulose having a moisture content as disclosed above is directly obtained by partial depolymerization of cellulose and subsequent washing, which process steps lead to a wet mass that is referred to in the art by several names, including hydrolyzed cellulose, hydrolyzed cellulose wetcake, level-off DP cellulose, microcrystalline cellulose wetcake or simply wetcake.

In another embodiment of the present invention, MCC and a liquid, such as water, can be mixed, such as kneaded, in a compounder to provide a microcrystalline cellulose having a moisture content as disclosed above.

The obtained moist MCC is subsequently subjected to mill-drying in a mill drying device, according to the process of the present invention. The compounder preferably allows thorough and intense mixing. Useful compounders are, for example, granulators, kneaders, extruders, presses, or roller mills, wherein the mixture of the MCC and liquid is homogenized by applying shear forces and compounding, such as a twin-screw compounder. Co-rotating as well as counter-rotating machines are suitable. So-called divided trough kneaders with two horizontally arranged agitator blades that engage deeply with one another and that perform a mutual stripping action, as in the case of twin-screw compounders are particularly suitable. Suitable single-shaft, continuous kneaders include the so-called Reflector® compounders, which are high performance mixers of modular construction, consisting of a multi-part, heatable and coolable mixing cylinder and a unilaterally mounted blade mixer (manufacturer: Lipp, Germany). Also suitable are so-called pinned cylinder extruders or Stiftconvert® extruders (manufacturer: Berstorff, Germany). The pins incorporated in the housing serve as abutments in order to prevent the kneaded material rotating together with the shaft. Kneader mixers with so-called double-blade sigma stirrers (manufacturer: Fima, Germany) in a horizontal assembly are particularly suitable. The blades operate at different speeds and their direction of rotation can be reversed. A stirred vessel with a vertically arranged mixer shaft is also suitable if suitable flow baffles are mounted on the vessel wall in order to prevent the kneaded mass rotating together with the stirrer shaft, and in this way an intensive mixing action is imparted to the kneaded material (manufacturer: Bayer AG). Also suitable are double-walled mixing vessels with a planetary stirrer and inline homogenizer.

In an embodiment of the invention the gas is fed into a gas-swept mill-drying device at a flow rate of from 1000 to 4000 m³/h, preferably from 1100 to 3000 m³/h, such as from 1200 to 2800 m³/h, such as from 1400 to 2600 m³/h, such as from 1500 to 2500 m³/h, such as from 1600 to 2300 m³/h.

In an embodiment of the invention the gas is fed into a gas-swept mill-drying device at a flow rate of from 10 to 1000 m³ gas/kg MCC_{dry}, preferably from 20 to 500 m³ gas/kg MCC_{dry}, more preferably from 75 to 230 m³ gas/kg MCC_{dry}.

In one aspect of the invention the circumferential speed of the mill-drying device, preferably the gas-swept impact mill, is preferably not more than 220 m/s, such as not more than 200 m/s, such as not more than 150 m/s, such as not more than 130 m/s or not more than 120 m/s. In an aspect of the invention the circumferential speed of the mill-drying device is preferably more than 20 m/s, such as more than 30 m/s, such as more than 40 m/s, such as more than 50 m/s. In an aspect of the invention the gas-swept mill-drying device is operated in such a manner that its circumferential speed is in a range from 30 to 130 m/s, more preferably from 50 to 120 m/s, such as from 60 to 120 m/s.

In one aspect of the invention the mill-drying device, preferably the the gas-swept impact mill, is operated at preferably not more than 20,000 rpm (revolutions per minute), such as not more than 15,000 rpm, such as not more than 8000 rpm. In an aspect of the invention the mill-drying device is operated at more than 1000 rpm, such as more than 1200 rpm, such as more than 1500 rpm.

In an embodiment of the present invention, the moisture content of the produced microcrystalline cellulose (MCC) after mill-drying is less than 20 percent, such as up to 15 percent, such as up to 10 percent, preferably up to 5 percent, more preferably up to 4 percent, such as from 1-4 percent, such as 1.5-4, such as 2-4 or such as 2.5-3.5 percent, based on the total weight of the produced MCC.

Particle size and shape (LEFI, DIFI and EQPC) of a particulate cellulose, such as MCC, can be determined by a high-speed image analysis method which combines particle size and shape analysis of sample images. An image analysis method for complex powders is described in: W. Witt, U. Köhler, J. List, Current Limits of Particle Size and Shape Analysis with High Speed Image Analysis, PARTEC 2007. A high-speed image analysis system is commercially available from Sympatec GmbH, Clausthal-Zellerfeld, Germany as dynamic image analysis (DIA) system QICPIC™. The system analyses the shape of the particles and takes potential curliness of the particles into account. It provides a more accurate measurement of true particle sizes (LEFI, DIFI and EQPC) than other methods. The dynamic image analysis (DIA) system QICPIC™ is described in more detail by Witt, W., Köhler, U., List, J.: Direct Imaging of very fast Particles Opens the Application of Powerful (dry) Dispersion for Size and Shape Characterization, PARTEC 2004, Nuremberg, Germany.” The high-speed image analysis system is useful for measuring among others the following dimensional parameters of particles:

The EQPC (Equivalent Projected Circle Diameter) of the particle is defined as the diameter of a circle that has the same area as the projection area of the particle. The EQPC (50,3) is the median diameter of a Circle of Equal Projection Area and is defined as follows: All particle size distributions, e.g. the EQPC can be displayed and applied as number (0), length (1), area (2) or volume (3) distribution. The volume distribution of the EQPC is calculated as cumulative distribution Qs. The volume distribution within the diameter of a Circle of Equal Projection Area value EQPC 50,3 is designated by the number 3 after the comma. The designation 50, reflecting the median value, stands for 50% of the EQPC of particle distribution being smaller than the given value in μm and 50% being larger. The 50% EQPC value is calculated by the image analyzer software. A high-speed image analysis system is commercially available from Sympatec GmbH, as dynamic image analysis (DIA) system QICPIC™, referred to above.

The microcrystalline cellulose that is produced according the process of the present invention generally has a median EQPC (EQPC 50,3) of at least 10 micrometers, preferably at least 20 micrometers, more preferably at least 30 micrometers, such as at least 40 micrometers, or such as at least 50 micrometers. The microcrystalline cellulose that is produced according the process of the present invention generally has a median EQPC (EQPC 50,3) of up to 400 micrometers, preferably up to 300 micrometers, more preferably up to 250 micrometers, such as up to 200 micrometers, such as up to 150 micrometers, such as up to 120 micrometers, such as up to 100 micrometers.

LEFI: The particle length LEFI is defined as the longest direct path that connects the ends of the particle within the contour of the particle. “Direct” means without loops or branches. For the purpose of the present invention the median LEFI is based on the volume distribution of all particles in a given sample of a particulate MCC. The median LEFI means that 50% of the LEFI of the particle distribution is smaller than the given value in μm and 50% is larger. The microcrystalline cellulose that is produced according the process of the present invention generally has a median LEFI of at least 10 micrometers, preferably at least 30 micrometers, more preferably at least 40 micrometers, such as at least 50 micrometers or such as at least 60 micrometers. The microcrystalline cellulose that is produced according the process of the present invention generally has a median LEFI of up to 400 micrometers, preferably up to 300 micrometers, more preferably up to 200 micrometers, such as up to 160 micrometers, such as up to 120 micrometers.

DIFI: The particle diameter is calculated by dividing the projection area by the sum of all lengths of the branches of the particle skeleton. DIFI is calculated automatically by the software PAQXOS of the dynamic image analysis (DIA) system QICPIC™. For the calculation of DIFI the software PAQXOS is applying this method to those particles only that are completely within the image frame. For the purpose of the present invention the median DIFI is based on the volume distribution of all particles in a given sample of a particulate MCC. The median DIFI means that 50% of the DIFI of the particle distribution is smaller than the given value in μm and 50% is larger.

The microcrystalline cellulose that is produced according the process of the present invention generally has a median DIFI of at least 10 micrometers, preferably at least 20 micrometers, more preferably at least 25 micrometers, such as at least 30 micrometers, or such as at least 40 micrometers. The microcrystalline cellulose that is produced according the process of the present invention generally has a median DIFI of up to 400 micrometers, preferably up to 300 micrometers, more preferably up to 200 micrometers, such as up to 100 micrometers, such as up to 90 micrometers, such as up to 80 micrometers, or such as up to 70 micrometers.

Bulk density (BD) as used herein is defined as the ratio of apparent volume to mass of the material taken, called untapped bulk density, and also the ratio of tapped volume to mass of material taken, called tapped bulk density. A useful procedure for measuring these bulk densities is described in United States Pharmacopeia 24, Test 616 “Bulk Density and Tapped Density,” United States Pharmacopeia Convention, Inc., Rockville, Maryland, 1999.

The microcrystalline cellulose that is produced according the process of the present invention generally has an untapped bulk density of at least 100 g/L, preferably of at least 200 g/L, more preferably of at least 350 g/L, and most preferably at least 400 g/L. In some embodiments of the invention, the microcrystalline cellulose even has an untapped bulk density of at least 450 g/L, or even at least 500 g/L. The microcrystalline cellulose that is produced according the process of the present invention generally has an untapped bulk density of up to 2000 g/L, preferably up to 1500 g/L, more preferably up to 1200 g/L, such as up to 1000 g/L, such as up to 900 g/L, such as up to 800 g/L. The microcrystalline cellulose that is produced according the process of the present invention generally has a tapped bulk density of at least 200 g/L, preferably of at least 300 g/L, more preferably of at least 400 g/L, most preferably at least 500 g/L, such as at least 600 g/L, such as at least 650 g/L or such as at least 700 g/L. The microcrystalline cellulose that is produced according the process of the present invention generally has a tapped bulk density of up to 2000 g/L, preferably up to 1500 g/L, more preferably up to 1200 g/L, such as up to 1000 g/L, such as up to 900 g/L.

The Carr index C is an indication of the compressibility of a powder. It is calculated by the formula C=100*(BD tapped−BD untapped)/BD tapped, wherein “BD tapped” is the tapped bulk density of a powder and “BD untapped” is the untapped bulk density of a powder. The Carr index is represented as a percentage. The Carr index is frequently used in the pharmaceutical science as an indication of the flowability of a powder. A Carr index of greater than 30 is usually an indication of poor flowability of a powder.

The microcrystalline cellulose that is produced according the process of the present invention generally has a Carr index of at least 10, such as of at least 12, such as of at least 14.

The microcrystalline cellulose that is produced according the process of the present invention generally has a Carr index of up to 31, such as of up to 30, preferably of up to 29, more preferably up to 28, such as of up to 27, such as 26, such as up to 25, such as up to 24 or such as up to 20.

In an embodiment the microcrystalline cellulose of the present invention and the microcrystalline cellulose that is produced according the process of the present invention or both have an untapped bulk density of at least 350 g/L or a Carr Index of up to 28 or both.

### Examples

### Comparative Examples

For comparative purposes, properties of two different grades of spray dried microcrystalline cellulose (MCC) were compared to properties of microcrystalline celluloses that were mill dried according to the process of the present invention. The two different grades of spray dried MCC differ in particle size and are commercially available under the trademark Avicel® PH101 and Avicel® PH102.

### General Description of Examples 1 to 16

Wetcake material used in Examples 1-10 and Examples 13-16 was obtained from a manufacturing process for producing commercial Avicel PH101 microcrystalline cellulose. The wetcake material had 63-65% moisture content, based on the total weight of the microcrystalline cellulose. Wetcake material was manually transferred to the dosing vessel (to reach minimum level required for continuous and stable feeding) located before a milling-drying unit. From the dosing vessel wetcake was transported continuously via feeding screw located at the bottom of the vessel. The material was forced through a perforated plate (die plate) (d=12-14 mm of voids; alternatively, for Examples 11-12 where feeding was performed without a perforated plate (die plate)); directly into the side of an Ultrarotor II “S” impact mill (Altenburger Maschinen Jaeckering GmbH, Hamm, Germany) between the first and second grinding stage. The mill was equipped with seven grinding stages Examples 9-13 were conducted with mill design 1 (‘fine design’) equipped with three “turbo” and four “standard” grinding bars and 12 sifters; Examples 1-8, 14-16 were conducted with mill design 2 (‘Coarse design’) equipped with seven “standard” grinding bars and without sifters. The rotor of the impact mill was operated at a circumferential speed up to 114 m/s (or 4444 rpm=100%). Variations of mill rotation speed are summarized in Examples 1-3. A specific gas flow system used herein was a closed loop system applying nitrogen (Examples 1-15) or steam (Example 16) as carrier and drying gas, variations of gas flow are summarized in Example 6-8. Temperature of the gas stream was controlled via a natural gas burner and a gas cooling system using cold water as coolant. The resulting gas temperatures of the respective gas streams are listed in Examples 1-16; variations of inlet temperature are listed in Examples 4-5. The microcrystalline cellulose samples were directly collected after the mill-drying step through an Algaier tumbler screening machine (Allgaier, Uhingen, Germany) equipped with a 500 μm sieve. The final product moisture was less than 6.3% by weight, based on the total weight of the colloidal microcrystalline cellulose.

The tapped (value was measured with 180× tapping) and untapped bulk density of the dried microcrystalline cellulose materials was measured using Hosokawa Powder Characteristics Tester: Model PT-S available from Hosokawa Micron, Osaka Japan. Values of Carr Index (as percentage) were calculated as “(Tapped bulk density—Untapped bulk density)/Tapped bulk density. 100”.

Particle size of dried microcrystalline cellulose samples, represented as median DIFI (X50), median LEFI (X50) and median EQPC (X50), were measured by an image analyzer (high speed image analyzer sensor QICPIC, Sympatec, Germany, with dry disperser RODOS/L with an inner diameter of 4 mm and dry feeder VIBRI/L and Software WINDOX5, Vers. 5.8.2.1 and M7 lens).

### Examples 11-12

A commercially available continuous compounder with heating and cooling jacket was used to add water to dry Avicel® PH101 microcrystalline cellulose commercially available from DuPont Chemical Company. The compounder jacket was supplied with a fluid of about 60° C. The fluid in the compounder jacket was used to adapt the temperature of the microcrystalline cellulose prior to drying and grinding to 25-60° C., but the temperature of the microcrystalline cellulose did not reach the temperature of the fluid in the compounder jacket because the water used for the hydration of the microcrystalline cellulose in the compounder was only at about 30° C. Examples 11-12 disclose the results with a compounder, with direct transport of freshly formed wetcake material to a dosing vessel located before the impact mill. The microcrystalline cellulose which was used in Examples 11-12 as a starting material had an untapped bulk density of about 290 g/l, a tapped bulk density of 428 g/I and Carr Index of 32.2. Particle size measured with above-mentioned QICPIC analyzer was the following: Median DIFI (X50)=42.5 μm, Median LEFI (X50)=132.9 μm and Median EQPC (X50)=76.2 μm. The microcrystalline cellulose wetcake (with moisture of 47-65%) which was freshly formed in the compounder was mill-dried as discussed above (Examples 1-10 and 13-16).

The conditions of the process of the present invention and the properties of the produced particulate microcrystalline cellulose are listed in Examples 1-16 below.

### Examples 1-3. Variations of Mill Rotation Speed (Rpm)

Gas inlet temperature: 90° C.; Flow through the mill: 2200 m³/h; Solid feed: 11 kg/h (value calculated on dry solid after milling-drying); Wetcake moisture: 65%;

Mill design: 2 (Coarse design); Dosing vessel design: with die plate; Drying gas: Nitrogen; No compounder.

TABLE 1

| | | Bulk | | | | | |
|---|---|---|---|---|---|---|---|
| | | Density | | | | | Moisture |
| | Mill | (untapped/ | | Median | Median | Median | after |
| | rpm | tapped | Carr | DIFI | LEFI | EQPC | mill-drying |
| Example | (%) | (g/L) | Index | (μm) | (μm) | (μm) | (%) |
| Comparative, | NA | 290/428 | 32.2 | 42.5 | 132.9 | 76.2 | <5 |
| Avicel ® PH101 | | | | | | | |
| (spray dried) | | | | | | | |
| Comparative, | NA | 322/454 | 29.1 | 63.5 | 187.8 | 121.1 | <5 |
| Avicel ® PH102 | | | | | | | |
| (spray dried) | | | | | | | |
| 1 | 50 | 663/798 | 16.9 | 92.2 | 190.25 | 138.7 | 4.6 |
| 2 | 70 | 648/799 | 19.0 | 60.9 | 109.2 | 88.5 | 3.8 |
| 3 | 100 | 552/750 | 26.4 | 41.7 | 66.35 | 56.9 | 2.3 |

Examples 4-5. Variations of gas inlet temperature (° C.): Flow through the mill: 2200 m³/h; Mill rpm: 100% (corresponding to 4444 rpm); Solid feed: 11 kg/h (value calculated on dry solid after milling-drying); Wetcake moisture: 65%; Mill design: 2 (Coarse design); Dosing vessel design: with die plate; Drying gas: Nitrogen; No compounder.

TABLE 2

| | | Bulk | | | | | |
|---|---|---|---|---|---|---|---|
| | Gas | Density | | | | | Moisture |
| | Inlet | (untapped/ | | Median | Median | Median | after |
| | T, | tapped) | Carr | DIFI | LEFI | EQPC | mill-drying |
| Example | ° C. | (g/L) | Index | (μm) | (μm) | (μm) | (%) |
| Comparative, | NA | 290/428 | 32.2 | 42.5 | 132.9 | 76.2 | <5 |
| Avicel ® PH101 | | | | | | | |
| (spray dried) | | | | | | | |
| Comparative, | NA | 322/454 | 29.1 | 63.5 | 187.8 | 121.1 | <5 |
| Avicel ® PH102 | | | | | | | |
| (spray dried) | | | | | | | |
| 4 | 75 | 569/760 | 25.2 | 43.8 | 74.4 | 60.9 | 3.2 |
| 5 | 105 | 537/743 | 27.7 | 40.6 | 63.9 | 55.3 | 1.6 |

Examples 6-8. Variations of gas flow through mill (m³/h): Gas inlet temperature: 75° C.; Mill rpm: 100% (corresponding to 4444 rpm); Solid feed: 11 kg/h (value calculated on dry solid after milling-drying); Wetcake moisture: 65%; Mill design: 2 (Coarse design); Dosing vessel design: with die plate; Drying gas: Nitrogen; No compounder.

TABLE 3

| | | Bulk | | | | | |
|---|---|---|---|---|---|---|---|
| | Flow | Density | | | | | Moisture |
| | through | (untapped/ | | Median | Median | Median | after |
| | mill | tapped) | Carr | DIFI | LEFI | EQPC | mill-drying |
| Example | (m³/h) | (g/L) | Index | (μm) | (μm) | (μm) | (%) |
| Comparative, | NA | 290/428 | 32.2 | 42.5 | 132.9 | 76.2 | <5 |
| Avicel ® PH101 | | | | | | | |
| (spray dried) | | | | | | | |
| Comparative, | NA | 322/454 | 29.1 | 63.5 | 187.8 | 121.1 | <5 |
| Avicel ® PH102 | | | | | | | |
| (spray dried) | | | | | | | |
| 6 | 1185 | 591/768 | 23.1 | 41.9 | 64.3 | 56.8 | 3.2 |
| 7 | 1606 | 572/767 | 25.4 | 42.4 | 65.6 | 57.6 | 3.5 |
| 8 | 2200 | 569/760 | 25.2 | 43.8 | 74.4 | 60.9 | 3.2 |

Examples 9-10 Variations of Solid feed (kg/h) (value calculated based on dry solid after milling-drying): Gas inlet temperature: 70-77° C.; Flow through the mill: 2000 m³/h; Mill rpm: 50% (corresponding to 2222 rpm); Wetcake moisture: 63.5%; Mill design: 1 (fine design); Dossing vessel design: with die plate; Drying gas: Nitrogen; No compounder.

TABLE 4

| | | Bulk | | | | | |
|---|---|---|---|---|---|---|---|
| | | Density | | | | | Moisture |
| | Solid | (untapped/ | | Median | Median | Median | after |
| | feed, | tapped) | Carr | DIFI | LEFI | EQPC | mill-drying |
| Example | kg/h | (g/L) | Index | (μm) | (μm) | (μm) | (%) |
| Comparative, | NA | 290/428 | 32.2 | 42.5 | 132.9 | 76.2 | <5 |
| Avicel ® PH101 | | | | | | | |
| (spray dried) | | | | | | | |
| Comparative, | NA | 322/454 | 29.1 | 63.5 | 187.8 | 121.1 | <5 |
| Avicel ® PH102 | | | | | | | |
| (spray dried) | | | | | | | |
| 9 | 9.2 | 688/824 | 16.5 | 86.5 | 155.8 | 127.3 | 4.9 |
| 10 | 16.3 | 707/827 | 14.5 | 101.8 | 192.8 | 153.4 | 6.3 |

Examples 11-12. Variations of Wetcake moisture (%): Gas inlet temperature: 115° C.; Flow through the mill: 2200 m³/h; Mill rpm: 100% (corresponding to 4444 rpm); Solid feed: 11 kg/h; Mill design: 1 (fine design); Dossing vessel design: without die plate; Drying gas: Nitrogen; Compounder: with compounder.

TABLE 5

| | | Bulk | | | | | |
|---|---|---|---|---|---|---|---|
| | | Density | | | | | Moisture |
| | Wetcake | (untapped/ | | Median | Median | Median | after |
| | moisture | tapped) | Carr | DIFI | LEFI | EQPC | mill-drying |
| Example | (%) | (g/L) | Index | (μm) | (μm) | (μm) | (%) |
| Comparative, | NA | 290/428 | 32.2 | 42.5 | 132.9 | 76.2 | <5 |
| Avicel ® PH101 | | | | | | | |
| (spray dried) | | | | | | | |
| Comparative, | NA | 322/454 | 29.1 | 63.5 | 187.8 | 121.1 | <5 |
| Avicel ® PH102 | | | | | | | |
| (spray dried) | | | | | | | |
| 11 | 47.8 | 481/697 | 30.9 | 30.8 | 47.2 | 41.7 | 1.4 |
| 12 | 64.1 | 481/689 | 30.2 | 30.8 | 48.8 | 42.1 | 1.5 |

Examples 13-14. Variations of mill design: Gas inlet temperature: 90° C.; Flow through the mill: 2000-2200 m³/h; Mill rpm: 50% (corresponding to 2222 rpm); Wetcake moisture: 65%; Solid feed: 10-11 kg/h (value calculated on dry solid after milling-drying); Dosing vessel design: with die plate; Drying gas: Nitrogen; No compounder.

TABLE 6

| | | Bulk | | | | | |
|---|---|---|---|---|---|---|---|
| | | Density | | | | | Moisture |
| | | (untapped/ | | Median | Median | Median | after |
| | Mill | tapped) | Carr | DIFI | LEFI | EQPC | mill-drying |
| Example | Design | (g/L) | Index | (μm) | (μm) | (μm) | (%) |
| Comparative, | NA | 290/428 | 32.2 | 42.5 | 132.9 | 76.2 | <5 |
| Avicel ® PH101 | | | | | | | |
| (spray dried) | | | | | | | |
| Comparative, | NA | 322/454 | 29.1 | 63.5 | 187.8 | 121.1 | <5 |
| Avicel ® PH102 | | | | | | | |
| (spray dried) | | | | | | | |
| 13 | 1 (fine | 650/802 | 19.0 | 75.2 | 136.7 | 111.0 | 3.2 |
| | design) | | | | | | |
| 14 | 2 (coarse | 663/798 | 16.9 | 92.2 | 190.2 | 138.7 | 4.6 |
| | design) | | | | | | |

Examples 15-16. Variations of Drying gas: Gas inlet temperature: 105-115° C.; Flow through the mill: 2200 m³/h; Mill rpm: 100% (corresponding to 4444 rpm); Solid feed: 11 kg/h (value calculated on dry solid after milling-drying); Wetcake moisture: 65%; Mill design: 2 (Coarse design); Dosing vessel design: with die plate; No compounder.

TABLE 7

| | | Bulk | | | | | |
|---|---|---|---|---|---|---|---|
| | | Density | | | | | Moisture |
| | | (untapped/ | | Median | Median | Median | after |
| | Drying | tapped) | Carr | DIFI | LEFI | EQPC | mill-drying |
| Example | gas | (g/L) | Index | (μm) | (μm) | (μm) | (%) |
| Comparative, | NA | 290/428 | 32.2 | 42.5 | 132.9 | 76.2 | <5 |
| Avicel ® PH101 | | | | | | | |
| (spray dried) | | | | | | | |
| Comparative, | NA | 322/454 | 29.1 | 63.5 | 187.8 | 121.1 | <5 |
| Avicel ® PH102 | | | | | | | |
| (spray dried) | | | | | | | |
| 15 | Nitrogen | 537/743 | 27.7 | 40.6 | 63.9 | 55.3 | 1.8 |
| 16 | Steam | 499/689 | 27.6 | 37.4 | 55.0 | 50.4 | 5.7 |

The results disclosed in the Tables 1-7 illustrate that mill-drying of microcrystalline cellulose according to the process of the present invention can provide mill-dried microcrystalline cellulose having similar particle size distribution (LEFI, DIFI and EQPC) and moisture content as the corresponding spray-dried microcrystalline cellulose. The results disclosed in Tables 1-7 also illustrate that according to the process of the present invention mill dried MCC can be produced that has taylor-made particle sizes (LEFI, DIFI and/or EQPC) according to the needs of the particular end-uses.

The results disclosed in the Tables 1-7 serve to illustrate that mill-drying of microcrystalline cellulose according to the process of the present invention can provide mill-dried microcrystalline cellulose having a higher tapped/untapped bulk density and/or a lower Carr index, while keeping a similar moisture content, as the corresponding spray-dried microcrystalline cellulose. Lower Carr index results in improved powder flow and higher bulk density results in improved packaging, storage and transportation.

Spray-dried microcrystalline cellulose is generally obtained by spray-drying a slurry of MCC having a moisture content of ca. 80 to 90 percent, based on the total weight of the slurry of MCC. The microcrystalline cellulose prepared according to the above examples is obtained by mill-drying moist MCC having a moisture content of ca. 50-65 percent. The mill-dried MCC of the present invention, exemplified in tables 1-7, is thus obtained with a substantially reduced consumption of water and energy as compared to spray-dried MCC.

### Examples 17-21

Wetcake material used in Examples 17-21 was obtained from a manufacturing process for producing commercial microcrystalline cellulose Avicel PH101. The wetcake material had 63-65% moisture content, based on the total weight of the microcrystalline cellulose. Wetcake material was manually transferred to a single screw powder feeder having a screw diameter of 48 mm and a rotational speed of screw of 5 rpm. The wetcake material was fed directly into the side, at the lower end, of an Ultrarotor type “15” impact mill (Altenburger Maschinen Jaeckering GmbH, Hamm, Germany). Nitrogen as carrier and drying gas was also fed to the Ultrarotor type 15 impact mill at the lower end of the mill.

For carrying out Examples 17-19 the original mill design was utilized which was equipped with seven grinding plates. In Examples 17-19 the wetcake material was fed between the first and second grinding plates.

For carrying out Example 20 only the grinding plate located at the top of the dry-grinding chamber was left in the mill. In Example 20 the wetcake material was fed well below the single grinding plate into the mill. This allowed partial drying of the moist MCC in the gas stream before the flow of gas stream and the MCC hit the grinding plate.

For carrying out Example 21 only two grinding plates, one located at the bottom and one located at the top of the dry-grinding chamber were left in the mill. In Example 21 the wetcake material was fed between the two grinding plates into the mill.

The rotor of the impact mill was operated at a speed of up to 14000 rpm. Variations of mill rotation speed are summarized in Table 8 below. Gas flow was 40 m3/h for all examples. A specific gas flow system used herein was a closed loop system applying nitrogen as carrier and drying gas. Temperature of the gas stream was controlled via a natural gas burner and a gas cooling system using cold water as coolant. Variation of the gas inlet temperature resulted in variation of the gas outlet temperature, i.e. the gas temperature after the mill. The resulting gas temperatures of the respective gas streams after the mill are listed in Table 8. The microcrystalline cellulose samples were directly collected after the milling-drying step. The final product moisture was less than 5% by weight in all Examples and in the two Comparative Examples. The results are listed in Table 8 below.

TABLE 8

| | | | Gas | | | | | |
|---|---|---|---|---|---|---|---|---|
| | | | Temper- | Bulk | | | | |
| | Number | | ature | Density | | | | |
| | of | | after the | (untapped/ | | Median | Median | Median |
| | grinding | Mill | mill | tapped) | Carr | DIFI | LEFI | EQPC |
| Example | plates | rpm | (° C.) | (g/L) | Index | (μm) | (μm) | (μm) |
| Comparative, | NA | NA | NA | 290/428 | 32.2 | 42.5 | 132.9 | 76.2 |
| Avicel ® PH101 | | | | | | | | |
| (spray dried) | | | | | | | | |
| Comparative, | NA | NA | NA | 322/454 | 29.1 | 63.5 | 187.8 | 121.1 |
| Avicel ® PH102 | | | | | | | | |
| (spray dried) | | | | | | | | |
| 17 | 7 | 11500 | 110 | 540/NA | NA | 37.5 | 57.7 | 50.8 |
| 18 | 7 | 11500 | 142 | 480/NA | NA | 33.7 | 51.2 | 45.3 |
| 19 | 7 | 14000 | 142 | 436/585 | 26.3 | 27.6 | 45.5 | 37.3 |
| 20 | 1 | 14000 | 105 | 450/NA | NA | 43 | 87 | 64.3 |
| 21 | 2 | 8000 | 110 | 569/773 | 25.5 | 68.8 | 204.4 | 137.5 |

NA: not assessed

The results in Tables 1-8 above illustrate that mill-drying of microcrystalline cellulose according to the process of the present invention is not only a more efficient process than spray drying but surprisingly also provides mill-dried microcrystalline cellulose having a higher tapped/untapped bulk density and/or a lower Carr index, while achieving a similar moisture content as the corresponding spray-dried microcrystalline cellulose. A higher tapped/untapped bulk density and/or a lower Carr index is achieved over a wide range of median particle sizes (DIF, LEFM and EQPC) which encompass the median particle sizes of the spray dried comparative MCC, Avicel® PH101 and Avicel® PH102.

1. A process for producing mill dried microcrystalline cellulose (MCC) comprising the steps of
a) providing MCC having a moisture content of from 20 to 75 percent, based on the total weight of the moist MCC; and
b) mill-drying the moist MCC in a single device capable of milling and drying in combination.

2. The process according to claim 1, wherein the produced mill-dried microcrystalline cellulose has a moisture content of less than 20% by weight, based on the total weight of the produced mill-dried MCC.

3. The process according to claim 1, wherein MCC provided in step a) has a moisture content of from 50 to 70%, based on the total weight of the moist MCC.

4. The process according to claim 1, wherein the produced mill-dried microcrystalline cellulose has an untapped bulk density of at least 350 g/L or a Carr Index of up to 28 or both.

5. The process according to claim 1, wherein the produced mill-dried microcrystalline cellulose has a median Equivalent Projected Circle Diameter (EQPC) of up to 400 micrometers.

6. The process according to claim 1, wherein the produced mill-dried microcrystalline cellulose has a median length of particle (LEFI) of up to 400 micrometers.

7. The process according to claim 1, wherein the produced mill-dried microcrystalline cellulose has a median diameter of particle (DIFI) of up to 400 micrometers.

8. The process according to claim 1, wherein the device capable of milling and drying in combination is a gas-swept impact mill.

9. The process according to claim 1, wherein the device capable of milling and drying in combination is a flash mill dryer.

10. The process according to claim 1, wherein MCC provided in step a) is obtained by mixing microcrystalline cellulose with water in a compounder.

11. Microcrystalline cellulose producible by the process according to claim 1.

12. Microcrystalline cellulose according to claim 11 having an untapped bulk density of at least 350 g/L or a Carr Index of up to 28 or both.

13. Microcrystalline cellulose having a moisture content of less than 20% by weight, based on the total weight of the microcrystalline cellulose including moisture, and having an untapped bulk density of at least 350 g/L or a Carr Index of up to 28 or both.

14. A food, beverage, pharmaceutical or personal care product comprising the microcrystalline cellulose according to claim 11.

15. A food, beverage, pharmaceutical or personal care product comprising the microcrystalline cellulose according to claim 13.
